(19) United States
(12) Patent Application Publication
(10) Pub. No.: US 2026/0007409 A1
(43) Pub. Date: Jan. 8, 2026

(54) Flow Diverter Stent Apparatus for Treating Intracranial Aneurysms

(71) Applicant: Naci Kocer, Istanbul (TR)

(72) Inventor: Naci Kocer, Istanbul (TR)

(21) Appl. No.: 19/259,128

(22) Filed: Jul. 3, 2025

Related U.S. Application Data
(60) Provisional application No. 63/668,406, filed on Jul. 8, 2024.

(51) Int. Cl. A61B 17/12; A61B 17/00; A61B 90/00

(52) U.S. Cl. CPC A61B 17/12118; A61B 17/12172; A61B 90/39; A61B 2017/00477; A61B 2017/00862; A61B 2017/00867; A61B 2017/1205; A61B 2090/3966

(57) ABSTRACT

A flow diverter stent apparatus having a substantially tubular body is disclosed. In at least one embodiment, the apparatus provides an at least one braided segment and an at least one laser cut segment engaged with one another in series so as to form an elongate flow diverter stent. During use of the apparatus, with the apparatus positioned within a catheter and moved to a target area of a patient's vascular system, each of the at least one braided segment and at least one laser cut segment is configured for moving into a shape memorized secondary shape capable of filling the target area as the apparatus exits the catheter.

### RELATED APPLICATIONS

This application claims priority and is entitled to the filing date of U.S. provisional application Ser. No. 63/668,406, filed on Jul. 8, 2024. The contents of the aforementioned application are incorporated herein by reference.

### BACKGROUND

The subject of this patent application relates generally to intravascular therapeutic devices and more particularly to a flow diverter stent apparatus for treating intracranial aneurysms.

Applicant hereby incorporates herein by reference any and all patents and published patent applications cited or referred to in this application.

By way of background, intracranial stents for arterial aneurysm treatment are mainly indicated for blister-dissecting, fusiform morphology, complex-shaped aneurysms with very wide necks, bifurcation aneurysms, and regrowth of aneurysm treated with other techniques. Stents can prevent the prolapsing of the intra-saccular material in the parent artery and help promote the healing reaction at the aneurysm segment. Flow diverter (“FD”) stents create thrombosis-occlusion of the aneurysm sac over time, preserving the incorporated arteries by having a healing reaction on it and creating a new hemodynamic balance in the parent artery.

Flow diverters currently available on the market are mainly designed with braided wire technology. The main disadvantages of such braided wire designs are foreshortening, opening problems, and not having an optimum apposition to the vessel wall in the vessel with a discrepancy in diameter. Those disadvantages can further lead to sizing and manipulation difficulties. Traditional flow diverters are also unable to treat aneurysms in the vicinity of bifurcations without covering the other branch of the bifurcation. The high mesh design of traditional flow diverters also prevents further intervention into the covered branch by the flow diverter. Thus, there remains a need for an improved flow diverter stent that is capable of treating a wide variety of intracranial aneurysms without suffering from the typical problems associated with traditional stents and flow diverters.

Aspects of the present invention solve these problems and provide further related advantages as described below, taken in conjunction with the accompanying drawings, which illustrate, by way of example, the principles of aspects of the invention.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a flow diverter stent apparatus having a substantially tubular body. In at least one embodiment, the apparatus provides an at least one braided segment and an at least one laser cut segment engaged with one another in series so as to form an elongate flow diverter stent. The at least one braided segment provides a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends. The at least one laser cut segment provides a single piece of laser cut material having shape memory and super-elastic properties so as to form said laser cut segment capable of being inserted into a catheter and having a proximal end, a distal end, and a scaffold extending therebetween. The scaffold has a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of the at least one laser cut segment. The proximal end of the at least one laser cut segment provides a plurality of engagement cells arranged circumferentially about the proximal end of said laser cut segment, each of the engagement cells configured for engagement with at least one strand of the at least one braided segment at the distal end of the at least one braided segment. During use of the apparatus, with the apparatus positioned within a catheter and moved to a target area of a patient's vascular system, each of the at least one braided segment and at least one laser cut segment is configured for moving into a shape memorized secondary shape capable of filling the target area as the apparatus exits the catheter.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a perspective view of an exemplary flow diverter stent apparatus, in accordance with at least one embodiment;

FIG. 2 is a perspective view of a further exemplary flow diverter stent apparatus, in accordance with at least one embodiment;

FIG. 3 is a perspective view of a still further exemplary flow diverter stent apparatus, in accordance with at least one embodiment;

FIG. 4 is a top plan view of the apparatus in a flattened state, in accordance with at least one embodiment;

FIG. 4A is a detailed view of the section defined by line 4A of FIG. 4;

FIG. 5 is a top plan view of a still further exemplary flow diverter stent apparatus in a flattened state, in accordance with at least one embodiment;

FIG. 6 is a perspective view of a still further exemplary flow diverter stent apparatus, in accordance with at least one embodiment; and

FIGS. 7-11 are diagrammatic views of the apparatus in use, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

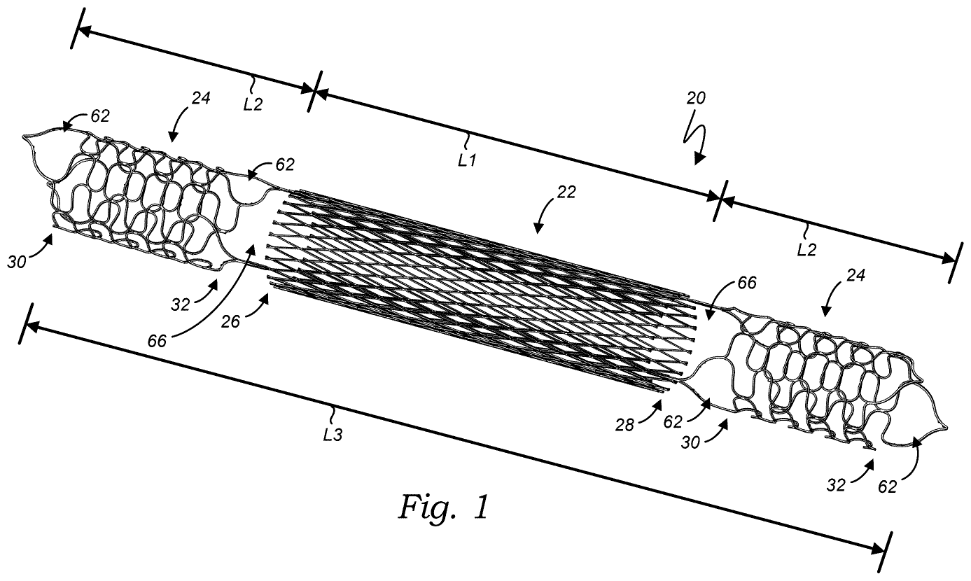

Turning now to FIG. 1, there is shown a perspective view of an exemplary flow diverter stent apparatus 20 for treating intracranial aneurysms in an unconstrained state, in accordance with at least one embodiment. In that regard, it should be noted that the unconstrained state of the apparatus 20 corresponds to no radial load and no longitudinal load being applied to the apparatus 20. Thus, the unconstrained state represents a condition where the apparatus 20 has no forces acting upon it, and is in an equilibrium or “memory” state. It should also be noted that the embodiments of the apparatus 20 depicted in the drawings are merely exemplary and are shown for illustrative purposes. Accordingly, in further embodiments, the apparatus 20 (along with each of the components of the apparatus 20 described herein) may take on any other sizes, shapes, dimensions and/or configurations now known or later developed—dependent at least in part on the specific context in which the apparatus 20 is to be utilized—so long as the apparatus 20 is able to substantially carry out the functionality described herein. Similarly, while the apparatus 20 is described herein in the context of intracranial aneurysms, in further embodiments, the apparatus 20 may be utilized in other body parts.

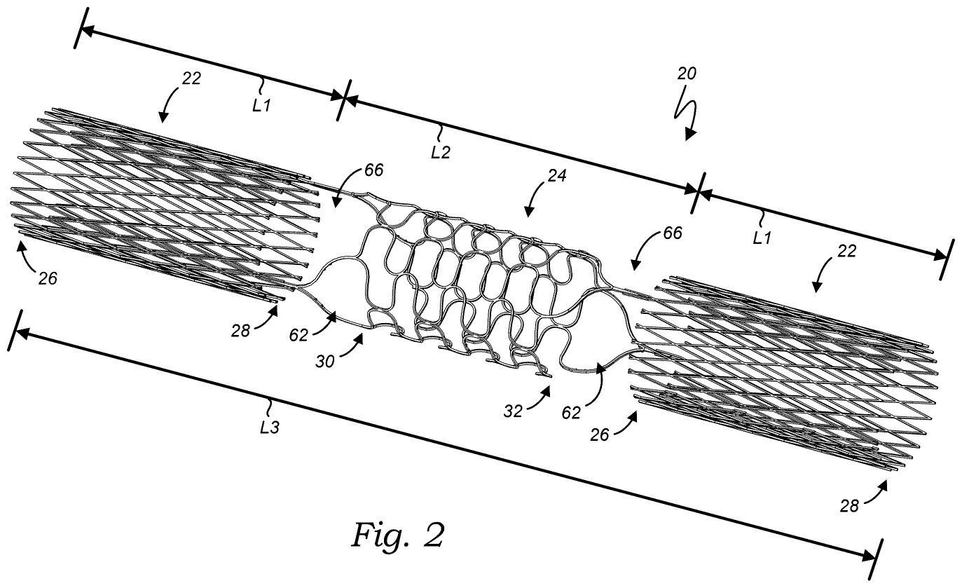

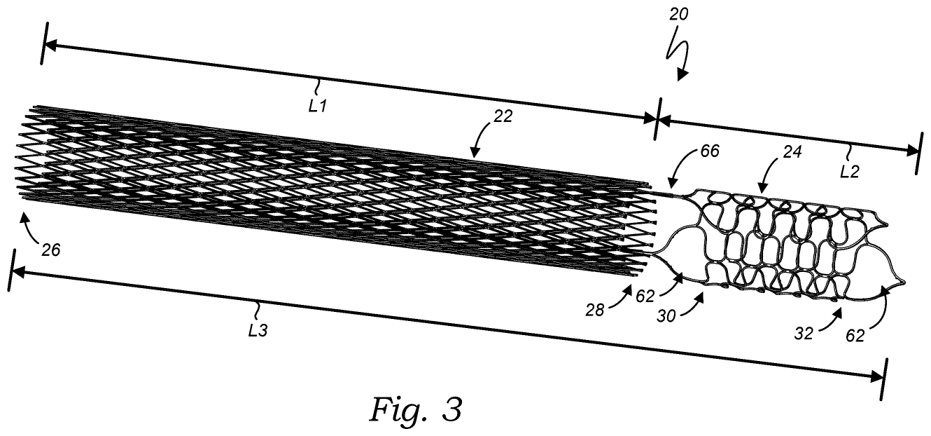

In at least one embodiment, the apparatus 20 provides an at least one braided segment 22 and an at least one laser cut segment 24 engaged with one another in series so as to form an elongate flow diverter stent. In at least one embodiment, as illustrated in FIG. 1, the apparatus 20 provides a single braided segment 22, with a proximal laser cut segment 24 engaged with a proximal end 26 of the braided segment 22, and a second laser cut segment 24 engaged with an opposing distal end 28 of the braided segment 22. In at least one such embodiment, each of the braided segment 22, first laser cut segment 24 and second laser cut segment 24 has a segment length L1 and L2 that is equal to approximately one-third (⅓) of an overall length L3 of the apparatus 20. In at least one alternate embodiment, as illustrated in FIG. 2, the apparatus 20 provides a single laser cut segment 24, with a first braided segment 22 engaged with a proximal end 30 of the laser cut segment 24, and a second braided segment 22 engaged with an opposing distal end 32 of the laser cut segment 24. In at least one such embodiment, each of the laser cut segment 24, first braided segment 22 and second braided segment 22 has a segment length L1 and L2 that is equal to approximately one-third (⅓) of the overall length L3 of the apparatus 20. In at least one further alternate embodiment, as illustrated in FIG. 3, the apparatus 20 provides a single braided segment 22, with a single laser cut segment 24 engaged with the distal end 28 of the braided segment 22. In at least one such embodiment, each of the braided segment 22 and laser cut segment 24 has a segment length L1 and L2 that is equal to approximately one-half (½) of the overall length L3 of the apparatus 20. In still further alternate embodiments, the apparatus 20 may provide any quantities of braided segments 22 and laser cut segments 24 in any ordered arrangement, now known or later developed (including, for example, the apparatus 20 depicted in FIG. 3, but reversed)—dependent at least in part on the specific context in which the apparatus 20 is to be utilized—so long as the apparatus 20 is able to substantially carry out the functionality described herein. Similarly, in further embodiments, each of the at least one braided segment 22 and at least one laser cut segment 24 may have any other segment length L1 and L2, now known or later developed—dependent at least in part on the specific context in which the apparatus 20 is to be utilized—so long as the apparatus 20 is able to substantially carry out the functionality described herein. Additionally, in further embodiments, the segment lengths L1 and L2 of the at least one braided segment 22 and at least one laser cut segment 24 may be substantially the same as or different than one another in a given embodiment of the apparatus 20.

In at least one embodiment, the at least one braided segment 22 is comprised of a plurality of individual strands 34 braided together so as to form the braided segment 22 having an elongated primary shape. It should be noted that the term “strand 34” is intended in its broadest meaning to include a wire, a fiber, a filament, or other single elongated member. Additionally, the term “radiopaque” is utilized for its normal meaning of being radiodense, that is, formed of one or more materials (such as platinum, chromium, cobalt, tantalum, tungsten, nitinol, gold, silver, stainless steel or alloys thereof, for example) which inhibit the passage of electromagnetic radiation to increase visibility during imaging. In at least one embodiment, one or both of the proximal and distal ends 26 and 28 of the at least one braided segment 22 are simply closed, sealed or otherwise finished so as to maintain the braided arrangement of the strands 34.

In at least one embodiment, given that the at least one braided segment 22 is comprised of a plurality of strands 34 braided together, the properties of the braided segment 22 may be selectively tailored to fit the requirements of the medical procedure for which the apparatus 20 is to be used. For example, in at least one embodiment, one or more individual strands 34 may be comprised of materials that are different from the other strands 34 of the braided segment 22. In at least one such embodiment, at least one of the strands 34 is comprised of one or more radiopaque materials, while the remaining strands 34 are comprised of one or more non-radiopaque materials (such as fiber, plastic, polymers, multi-layer composites or other biocompatible materials, for example). In this way, the radiopacity of the braided segment 22 may be selectively tailored (by including a lower or higher quantity of radiopaque strands 34) while also maintaining the requisite structural integrity for necessary shape retention. In at least one further embodiment, one or more individual strands 34 may be comprised of both radiopaque and non-radiopaque materials. Those skilled in the art will understand that other suitably bio-compatible materials may be used so long as they possess appropriate mechanical properties. Additionally, in at least one embodiment, one or more individual strands 34 has a strand diameter (i.e., an outer diameter) that is different than a strand diameter of the remaining strands 34 of the braided segment 22, which allows a primary diameter of the braided segment 22 (i.e., an outer diameter of the braided segment 22 while in the primary shape) to be selectively tailored which, in turn, can alter the mechanical performance of the braided segment 22. In at least one embodiment, the primary diameter is also dependent on the quantity of strands 34 as well as the braiding pattern utilized by the braided segment 22. The relative rigidity or softness of the braided segment 22 may also be selectively tailored based on one or more of the quantity of strands 34, the strand diameter of each strand 34, the material(s) of construction of each strand 34, and the braiding pattern utilized by the braided segment 22. In at least one embodiment, the at least one braided segment 22 can contain high mesh strands 34, low mesh strands 34, or a combination of both. In at least one embodiment, the braided segment 22 homogenously has high mesh strands 34 (for example, 48 strands 34), providing more metal coverage. Additionally, in at least one embodiment, half of the braided segment 22 has low mesh strands 34 (for example, 16 strands 34) proximally, and the other half of the braided segment 22 has high mesh strands 34 (48 strands 34) distally. In at least one alternate embodiment, the braided segment 22 always contain high mesh strands 34. In at least one embodiment, one or more individual strands 34 may be constructed out of, impregnated or coated with an at least one drug or drug releasing agent (hereinafter generally referred to as a “therapeutic” for simplicity purposes), such that the at least one therapeutic is capable of being automatically released into the patient's bloodstream when the apparatus 20 is deployed within the patient's vascular system. In at least one such embodiment, one or more strands 34 are coated with a therapeutic that promotes aneurysmal thrombosis.

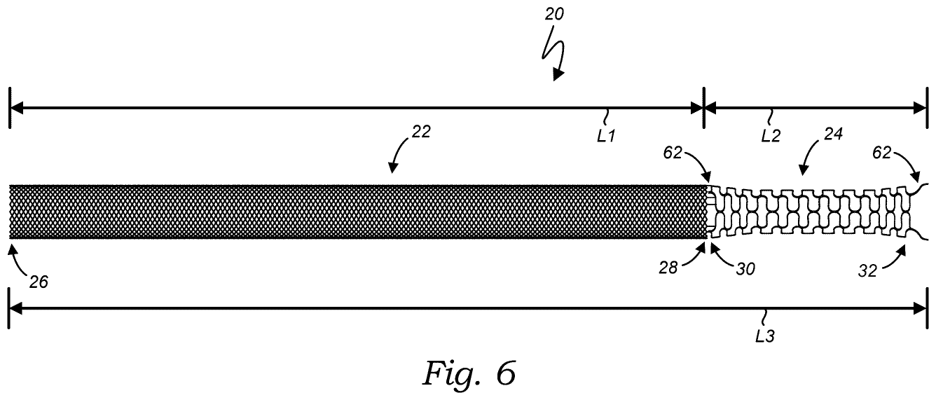

FIGS. 1-6 illustrate exemplary braiding patterns that the at least one braided segment 22 may utilize. The braided segment 22 has a braid density expressed in terms of picks per inch (“PPI”), which represents the number of times the strands 34 cross one another within a given inch of a length of the braided segment 22. Thus, the more times the strands 34 cross one another (i.e., the greater the PPI value), the greater the braid density. In at least one embodiment, as illustrated in FIGS. 1-6, the braided segment 22 defines a plurality of braid gaps 36 between strands 34 along the length of the braided segment 22. The dimensions and quantity of the braid gaps 36 is dependent upon one or more of the quantity of strands 34 utilized by the braided segment 22, the strand diameter of each strand 34, and the braid density. Thus, in further embodiments, the braided segment 22 may be configured for eliminating or at least minimizing the dimensions and quantity of the braid gaps 36. Additionally, in at least one embodiment, the strands 34 are arranged in a relatively lower density braid pattern that forms an open, substantially tubular central core extending between the proximal and distal ends 26 and 28 of the braided segment 22 and defining an inner diameter within the braided segment 22. In at least one alternate embodiment, the strands 34 may be arranged in a relatively higher density braid pattern that minimizes or eliminates the inner diameter of the braided segment 22. It should be noted that the specific braiding patterns illustrated in the accompanying figures are merely exemplary and are being shown and described for illustrative purposes only. In still further embodiments, the strands 34 of the braided segment 22 may be braided using any braid, knit, or weave patterns now known or later developed (the terms “braid” and “braided” as used herein intended to be all-encompassing for simplicity purposes)—including but in no way limited to 1-over-1-under-1, 1-over-2-under-2, 2-over-2-under-2, etc.—so long as the apparatus 20 is able to substantially carry out the functionality described herein. Similarly, the braid densities and braid gaps 36 depicted in the drawings are merely exemplary as well. In still further embodiments, the braided segment 22 may utilize any other braid densities, and the braid gaps 36 may take on any other sizes, shapes, quantities, dimensions or patterns, now known or later developed, so long as the apparatus 20 is able to substantially carry out the functionality described herein. Similarly, the strands 34 themselves may each take on any other sizes, quantities or dimensions, now known or later developed, so long as the apparatus 20 is able to substantially carry out the functionality described herein. For example, in at least one embodiment, each of the strands 34 may have a round shape, a flat shape, a square shape, a hexagonal shape, an oval shape, a ribbon shape, a hollow/tubular shape, etc. in cross-section.

In at least one embodiment, the at least one laser cut segment 24 is made from a single piece of laser cut material that exhibits shape memory and super-elastic properties, such as nitinol for example; however, in further embodiments, the at least one laser cut segment 24 may be constructed out of any other material (or combination of materials) now known or later developed, that allows the at least one laser cut segment 24 to substantially carry out the functionality described herein. In at least one embodiment, the at least one laser cut segment 24 includes a plurality of sections, a proximal end 30, a distal end 32, and a scaffold 38 comprising a plurality of radial support cells 40 and a plurality of flexible support cells 42. In at least one alternate embodiment, the scaffold 38 has a closed cell configuration with no radial support cells 40 or flexible support cells 42.

In at least one embodiment, the proximal end 30 of the at least one laser cut segment 24 has an open design instead of a closed design in order to provide a potential location of attachment to a corresponding delivery system or a braided segment 22. In at least one embodiment, the distal end 32 of the at least one laser cut segment 24 is similar in shape to a radial support cell 40, except that the distal end 32 terminates to rounded edges as opposed to attaching to continuous aspects of the at least one laser cut segment 24. In at least one embodiment, both proximal and distal ends 30 and 32 of the at least one laser cut segment 24 may terminate at the end of a repeating junction, such that the entire laser cut segment 24 body is symmetrical. In at least one alternate embodiment, one or both of the proximal and distal ends 30 and 32 of the at least one laser cut segment 24 may terminate with open designs to provide placement for additional radiopaque elements, attachments to a corresponding delivery system or a braided segment 22, or to provide additional atraumatic edges. In at least one embodiment, one or more of the radial support cells 40 and/or flexible support cells 42 may be constructed out of, impregnated or coated with an at least one drug or drug releasing agent (hereinafter generally referred to as a “therapeutic” for simplicity purposes), such that the at least one therapeutic is capable of being automatically released into the patient's bloodstream when the apparatus 20 is deployed within the patient's vascular system. In at least one such embodiment, one or more of the radial support cells 40 and/or flexible support cells 42 are coated with a therapeutic that promotes aneurysmal thrombosis.

In at least one embodiment, each radial support cell 40 of the at least one laser cut segment 24 is an arrangement of four struts 44, where the struts 44 are symmetrical along both an x-axis (longitudinal axis) and a y-axis (circumferential axis) of the laser cut segment 24. In at least one embodiment, the radial support cells 40 repeat in radial axial rows 46 along the length of the laser cut segment 24. Additionally, in at least one embodiment, each flexible support cell 42 is an arrangement of four struts 44, with the flexible support cells 42 repeating in flexible axial rows 48, adjacent to the radial axial rows 46 of radial support cells 40, along the length of the laser cut segment 24. In at least one embodiment, each flexible support cell 42 has mirror symmetry to a corresponding flexible support cell 42 in another flexible axial row 48, in a circumferential direction, of flexible support cells 42. In at least one embodiment, the radial axial rows 46 of the radial support cells 40 alternate with the flexible axial rows 48 of flexible support cells 42 circumferentially around the laser cut segment 24. In at least one embodiment, each radial support cell 40 is staggered or offset along the circumferential axis such that the adjacent radial axial rows 46 of radial support cells 40 do not share an aligned center axis. This staggered arrangement ensures that the laser cut segment 24 will deploy in a continuous manner, with each column of radial support cells 40 deploying along with adjacent radial support cells 40. Additionally, in at least one embodiment, each radial support cell 40 within a radial axial row 46 is aligned in a linear design along a line that is parallel to the central axis of the laser cut segment 24, and therefore does not have any angle or helical structure. As a result, in such embodiments, the laser cut segment 24 will simply bend without any twisting motion and thereby minimize any relative motion against the vessel wall 50.

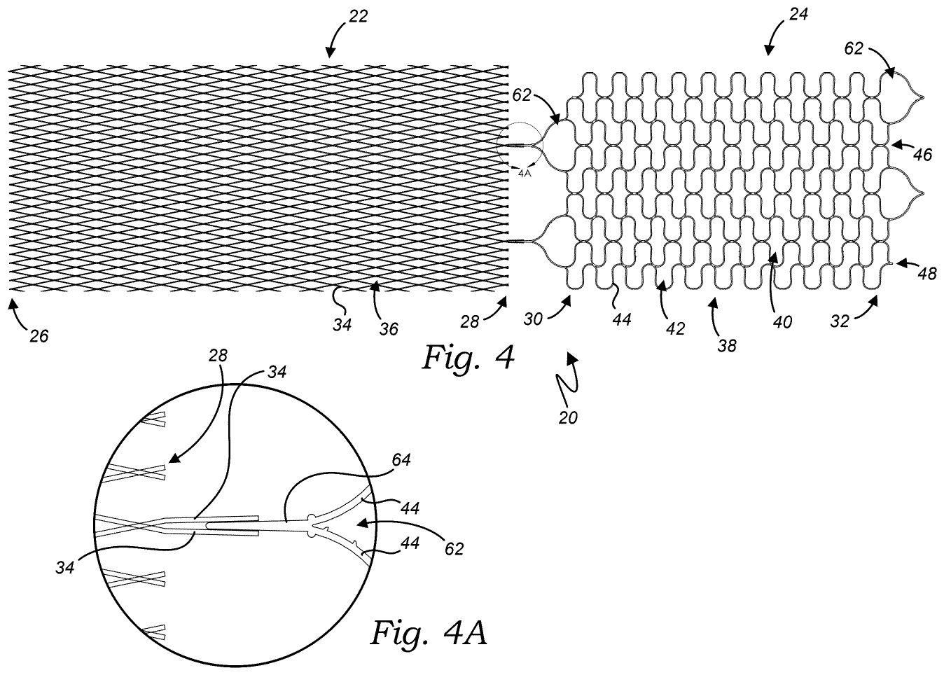

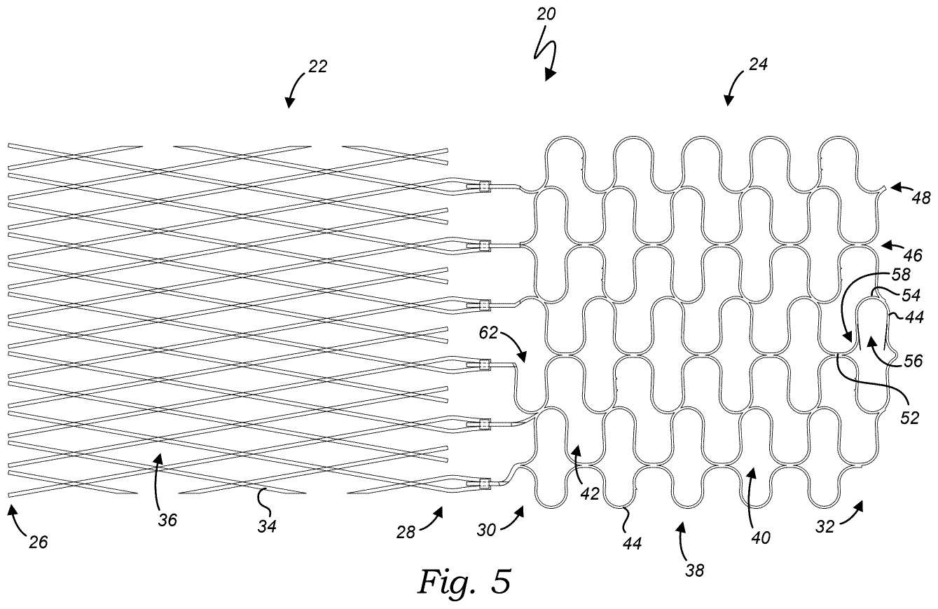

In at least one embodiment, each radial support cell 40 includes four vertical struts 44 and defines a joint apex 52, a top radius 54, and a vertical strut angle 56. In at least one embodiment, each vertical strut 44 is in a substantially vertical or less than vertical configuration while in an unconstrained or unloaded state, as shown in FIGS. 4 and 5. While under the unconstrained configuration, each radial support cell 40 is capable of adapting to different types of applied loads, such as a radial compression or load that is observed in a blood vessel, or an axial tensile stress that is observed while loading the at least one laser cut segment 24 into a delivery system. In the unconstrained configuration, the geometry of each radial support cell 40 (including the joint apex 52 and the top radius 54) has gentle radius edges to further support flexibility and conformability to the tubular nature of blood vessels 60.

In at least one embodiment, the radial support cells 40 and flexible support cells 42 of the at least one laser cut segment 24 are formed by uninterrupted material so as to form a laser cut segment 24 of unitary construction. In at least one alternate embodiment, adjacent ones of the radial support cells 40 and flexible support cells 42 are interrupted by an intentional break therebetween so as to create a non-continuous geometry. In at least one such embodiment, the break is then reconnected through the use of a coil or other permanent engagement mechanism, material or technique. Such a connection via the coil allows for multi-axial movement of the joint to maximize laser cut segment 24 flexibility when positioned within a target treatment vessel. In at least one embodiment, each coil is constructed out of a tungsten-loaded polymer. In further embodiments, the each coil may be constructed out of platinum, platinum iridium, chromium, cobalt, tantalum, nitinol, a nitinol composite, gold, silver, bismuth subcarbonate, barium sulfate, bismuth oxychloride, bismuth trioxide, stainless steel or alloys thereof, or any other radiopaque material (or combination of materials) now known or later developed.

FIGS. 4 and 5 show a flattened view of the apparatus 20 of FIG. 3 in an unconstrained state (which, in its final form, would be wrapped into a cylindrical/tubular design, similar to that shown in FIGS. 1-3). In at least one embodiment, when a radial load or radial force acts upon the at least one laser cut segment 24 (i.e., when the at least one laser cut segment 24 is in a radially constrained state), opposing vertical struts 44 of each radial support cell 40 buckle inwardly toward one another. In contrast to the radially constrained state, in the unconstrained state shown in FIGS. 4 and 5, opposing vertical struts 44 of each radial support cell 40 are substantially perpendicular to a central axis of the radial axial row 46 of radial support cells 40. Further, as the central axes of the radial axial rows 46 are parallel to the longitudinal axis of the laser cut segment 24, the vertical struts 44 are also substantially perpendicular to the longitudinal axis of the laser cut segment 24 in the unconstrained state. In at least one alternate embodiment, the vertical struts 44 of each radial support cell 40 are less than perpendicular with a vertical strut angle 56 of less than 0° in an unconstrained state to promote buckling while under a radial load or radial force. In at least one embodiment, when an axial tension or longitudinal force acts upon the at least one laser cut segment 24, the vertical struts 44 of each radial support cell 40 deform outwardly from one another. In at least one embodiment, the flexible support cells 42 of the at least one laser cut segment 24 exist as an axial (horizontal) row of cells to separate the radial support cells 40. Additionally, in at least one embodiment, the shape of the flexible support cells 42 permits the adjacent radial support cells 40 to be spaced apart and offset from one another in the circumferential direction, which further improves the flexibility of the laser cut segment 24 while maintaining radial strength. In at least one embodiment, each of the radial support cells 40 have the same size, shape and dimensions; however, in at least one alternate embodiment, one or more of the radial support cells 40 have a different size, shape and/or dimensions. Similarly, in at least one embodiment, each of the flexible support cells 42 have the same size, shape and dimensions; however, in at least one alternate embodiment, one or more of the flexible support cells 42 have a different size, shape and/or dimensions. Accordingly, in further embodiments, the radial support cells 40 and flexible support cells 42 may each take on any other sizes, shapes, dimensions, quantities, arrangements and/or configurations now known or later developed—dependent at least in part on the specific context in which the apparatus 20 is to be utilized—so long as the apparatus 20 is able to substantially carry out the functionality described herein.

In at least one embodiment, when the apparatus 20 is implanted into a blood vessel, the blood vessel will radially compress the apparatus 20. The continued radial force exerted by the apparatus 20 on the blood vessel prevents the blood vessel from collapsing or occluding, ensuring that blood may flow freely down the treated vessel. The continued radial force exerted by the apparatus 20 on the blood vessel also ensures that the apparatus 20 will properly adhere to the vessel wall to prevent movement both during the implantation procedure and post implantation. In at least one embodiment, as the apparatus 20 is radially compressed by the blood vessel, the vertical struts 44 of each radial support cell 40 of the at least one laser cut segment 24 will begin to buckle inwards, creating a vertical strut angle 56 of less than 0°. This buckling action increases the radial strength of the at least one laser cut segment 24. As the radial strength is derived from the buckling action, the radial strength is high in relation to the geometry of the individual vertical struts 44. As such, to align the at least one laser cut segment 24 with commonly accepted radial strength values, the stent geometry (e.g., wall thickness, strut 44 width) can be proportionately reduced. By reducing the stent geometry dimensions, the overall crossing profile may be reduced. Additionally, the footprint of the at least one laser cut segment 24 on the treatment vessel will be reduced, which may accelerate healing in vivo.

In at least one embodiment, when a given radial support cell 40 of the at least one laser cut segment 24 is in the radially loaded configuration, all aspects of the geometry remain rounded, with the top radius 54 and a vertical strut radius 58 maximized to reduce internal stress and strain within the design, and therefore minimize the risk of high stress failure. Conversely, the strong radial strength provided by the radial support cells 40 may be countered by applying a tensile load along an axis of two opposing joint apexes 52. This allows the apparatus 20 to be loaded into a delivery system without the need to combat the high radial strength observed during the buckling. In at least one embodiment, the radial support cells 40 of the at least one laser cut segment 24 may have varying vertical strut angles 56 ranging from about 45° to about 99°, as increasing amounts of axial tension or longitudinal force is applied to the radial support cells 40. As a tensile load is applied to the laser cut segment 24, the vertical strut angle 56 will increase, and in turn, the radial strength provided by the radial support cell 40 will decrease. This allows a simple and relatively low stress method of loading the apparatus 20 into a sheath or delivery system. In the tensile loaded configuration in at least one embodiment, all aspects of the geometry remain rounded, with the bulk of the vertical strut 44 movement captured within the top radius 54. Thus, in at least one embodiment, the vertical strut angle 56 plays a critical role in dramatically increasing both the radial strength characteristics and the flexibility of the at least one laser cut segment 24. In at least one embodiment, the vertical strut angle 56 is designed to be variable based upon the type of load applied to the laser cut segment 24, giving different loading conditions for radial and longitudinal loading. When under radial compression, the vertical struts 44 will buckle or invert, drastically increasing radial force. While under longitudinal loading, the vertical struts 44 will extend, drastically decreasing radial force.

Thus, in at least one embodiment, the at least one laser cut segment 24 reacts differently to longitudinal and radial loads, having a response optimized for each condition. Longitudinal loads are typically applied during loading or retraction, where the desired radial force is low. Radial loads are typically applied after the at least one laser cut segment 24 has reached its intended treatment location and is in contact with the vessel wall, where the desired radial force is high. In at least one embodiment, under radial loading, the vertical struts 44 will invert or buckle, resulting in a negative vertical strut angle 56, thereby increasing the laser cut segment 24 radial force dramatically. In this manner, in at least one embodiment, the at least one laser cut segment 24 provides a mechanism to give high radial force under radial load and low radial force under longitudinal loads.

In at least one embodiment, the apparatus 20 provides an at least one radiopaque element (e.g., a marker) positioned and configured for improving visibility and ease of use during an insertion or deployment procedure. In at least one embodiment, the at least one radiopaque element is positioned on one or both of a proximal end and a distal end of the apparatus 20—or, alternatively, on one or both of a proximal end 26 or 30 and a distal end 28 or 32 of one or more of the at least one braided segment 22 and laser cut segment 24. In at least one alternate embodiment, the at least one radiopaque element is positioned elsewhere along the length of the apparatus 20. In at least one embodiment, the at least one radiopaque element is constructed out of a tungsten-loaded polymer. In further embodiments, the at least one radiopaque element may be constructed out of platinum, chromium, cobalt, tantalum, nitinol, gold, silver, bismuth subcarbonate, barium sulfate, bismuth oxychloride, bismuth trioxide, stainless steel or alloys thereof, or any other radiopaque material (or combination of materials) now known or later developed.

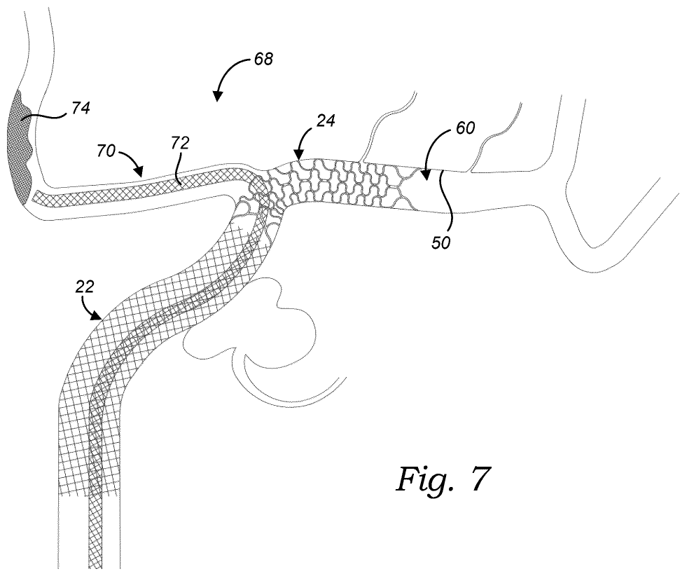

In at least one embodiment, a proximal end of the apparatus 20 (i.e., the proximal end 26 of the at least one braided segment 22 or the proximal end 30 of the at least one laser cut segment 24, depending on the ordered arrangement of the components) is removably engageable with a delivery mechanism (not shown), such as a wire for example, for moving the apparatus 20 through a catheter. In at least one embodiment, a distal end of the apparatus 20 (i.e., the distal end 28 of the at least one at least one braided segment 22 or the distal end 32 of the at least one laser cut segment 24, depending on the ordered arrangement of the components) incorporates a second guide wire. In at least one alternate embodiment, the second guide wire may be omitted. In at least one embodiment, the elongated primary shape of the apparatus 20 is suitable for insertion through the catheter into the vascular system with minimal discomfort to the patient. In at least one such embodiment, while in the primary shape, the apparatus 20 has the shape of an elongated bundle of strands 34, as discussed above. Once the apparatus 20 has reached a target area of the patient's vascular system, it may be deployed to its operative configuration and released from the delivery mechanism so that it will remain in position at the proper location. As illustrated in FIGS. 7-9, once in its operative configuration, the apparatus 20 takes on a secondary shape. Exemplary secondary shapes of the apparatus 20 include helices, vortices, flat spirals, complex spirals, three-dimensional complex shapes, spherical, or any other shape—now known or later developed—capable of effectively filling the affected blood vessel, as required by any specific clinical application. Additionally, in at least one embodiment, the secondary shape of the apparatus 20 may have a variable secondary diameter (i.e., an outer diameter of the apparatus 20 while in the secondary shape) and/or a variable pitch to fit into and to maintain its position in a specified space within the vascular system. In at least one alternate embodiment, the secondary shape of the apparatus 20 may have a uniform secondary diameter and/or a uniform pitch. The specific details of the size and shape of the secondary shape thus can be selected to fit the requirements of the medical procedure for which the apparatus 20 is to be used. Accordingly, the sizes, shapes and dimensions of each of the primary shapes and secondary shapes depicted in the accompanying figures are merely exemplary and shown for illustrative purposes. Additionally, any appropriate methods or techniques for setting the secondary shape of the apparatus 20, now known or later developed, may be utilized, such as use of high heat or memory shape material, for example.

As mentioned above, in at least one embodiment, the at least one braided segment 22 and at least one laser cut segment 24 are engaged with one another in series. In at least one such embodiment, as best illustrated in FIG. 4A, the proximal end 30 of the at least one laser cut segment 24 is engaged with the distal end 28 of the at least one braided segment 22 (or, alternatively, the proximal end 26 of the at least one braided segment 22 is engaged with the distal end 32 of the at least one laser cut segment 24) using weaving, welding (which may be single or double welding), brazing, soldering, gluing, crimping, or any other engagement mechanism or technique, now known or later developed. In at least one embodiment, one or both of the proximal end 30 and distal end 32 of the at least one laser cut segment 24 provides a plurality of engagement cells 62 arranged circumferentially about the associated proximal end 30 or distal end 32 of the at least one laser cut segment 24 (hereinafter referred to as an engagement end of the laser cut segment 24 for simplicity purposes). In at least one embodiment, each of the engagement cells 62 is an arrangement of four struts 44, where the struts 44 are symmetrical along both the x-axis (longitudinal axis) and y-axis (circumferential axis) of the laser cut segment 24. Additionally, the struts 44 of each of the engagement cells 62 define a joint apex 64 that is oriented longitudinally relative to the laser cut segment 24.

In at least one embodiment, the joint apex 64 of each engagement cell 62 is configured for engagement with one or more of the strands 34 of the corresponding proximal end 26 or distal end 28 of the at least one braided segment 22 (hereinafter referred to as an engagement end of the braided segment 22 for simplicity purposes). In at least one such embodiment, the joint apex 64 of each engagement cell 62 is positioned so as to be in overlapping contact with a corresponding at least one strand 34 of the engagement end of the braided segment 22, and the joint apex 64 and corresponding at least one strand 34 are then secured to one another using weaving, welding, brazing, soldering, gluing, crimping, or any other engagement mechanism or technique, now known or later developed. Thus, in at least one embodiment, the joint apex 64 of each engagement cell 62 is oriented at substantially the same angle as the corresponding at least one strand 34 of the engagement end of the braided segment 22, such that that the joint apex 64 and the corresponding at least one strand 34 can be joined without having to manipulate the corresponding at least one strand 34. In at least one embodiment, the joint apex 64 and corresponding at least one strand 34 are secured to one another through the use of a coil or other permanent engagement mechanism, material or technique. Such a connection via the coil allows for multi-axial movement of the joint to maximize apparatus 20 flexibility when positioned within a target treatment vessel. In at least one embodiment, each coil is constructed out of a tungsten-loaded polymer. In further embodiments, the each coil may be constructed out of platinum, platinum iridium, chromium, cobalt, tantalum, nitinol, a nitinol composite, gold, silver, bismuth subcarbonate, barium sulfate, bismuth oxychloride, bismuth trioxide, stainless steel or alloys thereof, or any other radiopaque material (or combination of materials) now known or later developed.

In at least one embodiment, each of the engagement cells 62 have the same size, shape and dimensions. In at least one alternate embodiment, one or more of the engagement cells 62 have a different size, shape and/or dimensions. In at least one embodiment, the engagement cells 62 are arranged circumferentially about the engagement end of the laser cut segment 24 so as to be equally spaced apart and symmetrical about the engagement end of the laser cut segment 24. In at least one alternate embodiment, the engagement cells 62 are arranged so as to be unequally spaced apart and asymmetrical about the engagement end of the laser cut segment 24. In at least one embodiment, the engagement cells 62 are sized and configured for creating a segment gap 66 between the engagement end of the laser cut segment 24 and the engagement end of the corresponding braided segment 22. In at least one alternate embodiment, the engagement cells 62 are sized and configured for positioning the engagement end of the laser cut segment 24 in substantially abutting contact with the engagement end of the corresponding braided segment 22. Accordingly, in further embodiments, the engagement cells 62 may take on any other sizes, shapes, dimensions, quantities, arrangements and/or configurations now known or later developed-dependent at least in part on the specific context in which the apparatus 20 is to be utilized-so long as the apparatus 20 is able to substantially carry out the functionality described herein.

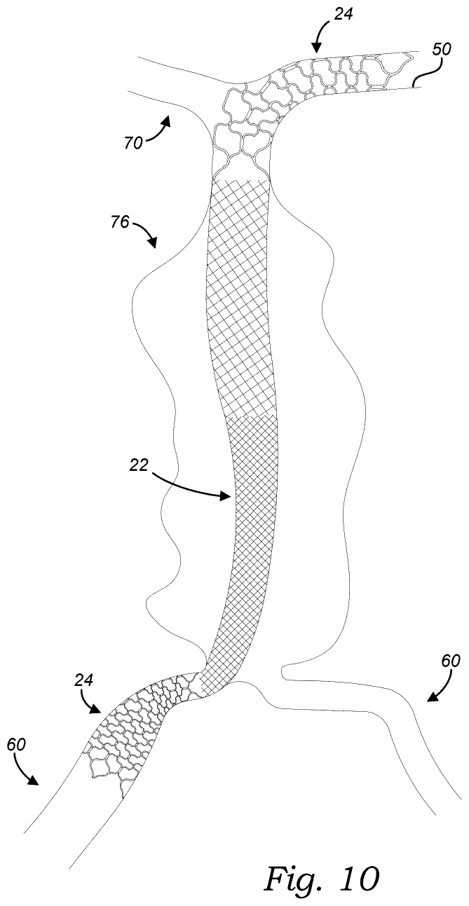

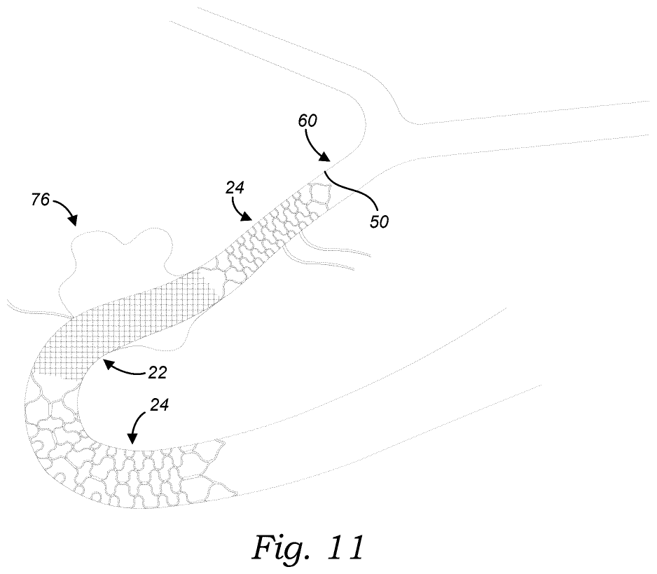

In at least one embodiment, the combination of braided segments 22 and laser cut segments 24 acts as a whole by facilitating each other's mechanical properties, especially when opening into an aneurysmal segment of the parent artery. The at least one laser cut segment 24 (distally or proximally) eliminates well-known opening problems of braided stents. The longer the braided stents, the more opening difficulties they can create, especially on the tight curves of the artery 68. Due to the nature of human vessels, getting thinner distally forces medical professionals to choose oversized flow diverter stents to adapt proximally to have better apposition, further preventing endoleak. Thus, in at least one embodiment, as illustrated in FIGS. 10 and 11, the apparatus 20, which combines a laser cut design with braided strands 34, aims to use all the advantages of both technologies. Those features are: no second-proximal opening problem without losing flow diverter effect; minimum foreshortening; better and precise opening and sizing; better apposition; and less manipulation. The apparatus 20 also provides a better solution to the bifurcation. Navigation into the covered side branch and aneurysm sac is still possible (Y stent option or stroke management). The pore size of the laser cut segment 24 will allow the passage of large profile microcatheters such as up to 0.021-0.027 inches, for example, for further intervention—though in further embodiments, the pore size of the laser cut segment 24 may allow for the passage of microcatheters smaller than 0.021 inches or larger than 0.027 inches. The apparatus 20 also allows less metal coverage in the perforating-rich segments of the artery. Accordingly, as illustrated in FIGS. 7-11, the apparatus 20 will help to reduce the fear of covering important arteries on the bifurcations.

The combined braided and laser cut technology is unique for single-layer stent design. In at least one embodiment, junction points, as shown in the accompanying drawing figures, do not create problems because available wire technology not only makes it happen but also enhances the visibility of the wires, either by adding markers to the apparatus 20 or by using drawin-filled tube (“DFT”) wires. So, visibility will not be a problem. Physicians will use the same preparation and deployment technique as they do with other stents with far better confidence. Using the same type of microcatheter (low profile for second aneurysm, higher profile for proximal ones), this unique new generation stent will make the deployment technique much easier and smoother with less manipulation. New adjunctive techniques such as surface modification and biodegradability (whole or partly) can be applied to it without a problem.

In at least one embodiment, by reducing the segment length L1 of the at least one braided segment 22 hemodynamically, the high mesh flow diverter part acts at the aneurysmal neck, especially at the inflow zone. The at least one laser cut segment 24 has a limited flow diverter effect, the purpose for which is to scaffold the whole segment of the parent artery either distally or proximally to the aneurysm by carrying the braided part into the aneurysmal neck. This lets the medical professional deploy less metal coverage at the non-diseased segment, especially at the bifurcation and high perforating rich areas. This assembly is very useful in the bifurcation aneurysm treatment to not cover the other branch with a high mesh braided segment 22. Oversizing of the at least one laser cut segment 24 is much easier on production with having fewer opening problems. The at least one laser cut segment 24 has a bigger pore size, which does not hinder the manipulation into the jailed ones and has fewer problems in the perforating rich areas. The at least one laser cut segment 24 allows the medical professional to reenter into the covered branch and aneurysm (in certain morphologies, which is the braided part that covers the inflow zone but partly covers the neck), as illustrated in FIGS. 7-9. In at least one embodiment, the design of the apparatus 20 is suitable for all aneurysms which had flow diverter indication. The size of the stents can be similar to flow diverter stents in the market. The bigger-diameter ones have longer versions than the smaller ones. The laser cut segment 24 can be slightly oversized, as illustrated in FIGS. 10 and 11.

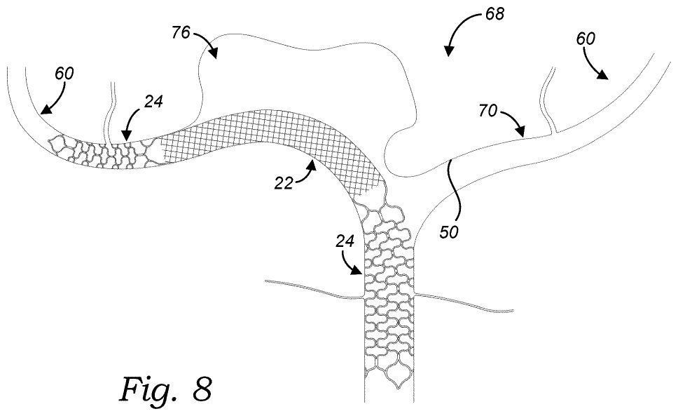

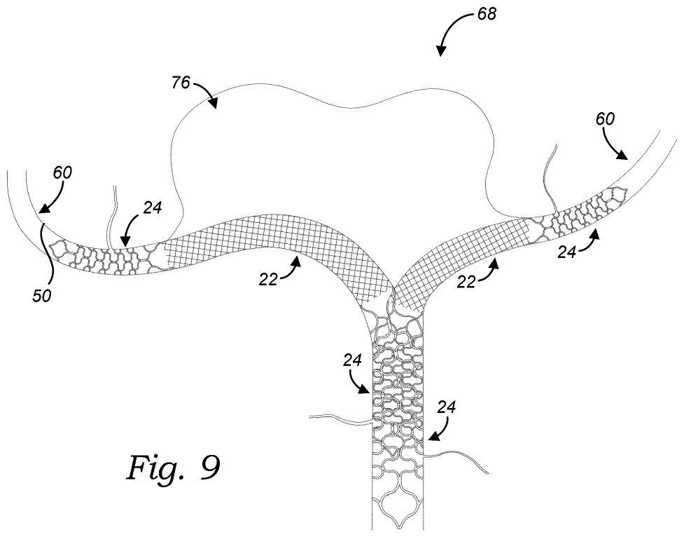

FIGS. 7-9 illustrate exemplary embodiments of the apparatus 20 in use in various contexts. In a bit more detail, FIG. 7 illustrates an exemplary use of the apparatus 20 in the context of performing a thrombectomy. In at least one such embodiment, if any thrombotic complication occurs in a jailed branch 70 of the patient's artery 68, any thrombectomy catheter 72, including large-bore aspiration catheters, are able to axially pass a distance through the apparatus 20 and subsequently exit the apparatus 20 via one of the radial support cells 40 of the at least one laser cut segment 24 in order to reach the thrombus 74. Accordingly, in such embodiments, one or more of the radial support cells 40 of the at least one laser cut segment 24 is large enough to allow a catheter 72 to pass therethrough. FIG. 8 illustrates an exemplary use of the apparatus 20 in the context of treating a basilar tip aneurysm 76 within an asymmetric bifurcation of the patient's artery 68. In at least one such embodiment, a single instance of the apparatus 20 can effectively resolve the issue. While the at least one braided segment 22 covers the neck of the aneurysm 76 (including the inflow zone), fewer laser-cut segments in the at least one laser cut segment 24 cover areas abundant in high perforating vessels. Therefore, the shortest possible high metal braided segment 22 will modify the hemodynamics at the neck of the aneurysm 76, while the at least one laser cut segment 24 functions as both a carrier of the at least one braided segment 22 into the diseased section of the parent artery 68 and acts as a scaffolding stent. FIG. 9 illustrates an exemplary use of the apparatus 20 in the context of treating a basilar aneurysm 76 within a wide neck symmetrical bifurcation of the patient's artery 68. In at least one such embodiment, where both posterior cerebral artery (“PCA”) segments have aneurysm necks, in order to achieve an optimal treatment model, “Y” stenting using two instances of the apparatus 20 can be a highly effective option. This is the first time such a “Y” stenting option through a flow diverter stent has been possible in such complex bifurcation models, thanks to the above-described aspects of the apparatus 20. Specifically, in at least one such embodiment, one or more of the radial support cells 40 of the at least one laser cut segment 24—or, alternatively, one or more segment gaps 66—is large enough to allow at least one further instance of the apparatus 20 (or a different type of stent) to pass therethrough. Thus, in at least one embodiment, the apparatus 20 is capable of treating a wide variety of intracranial aneurysms.

Aspects of the present specification may also be described as the following embodiments:

1. A flow diverter stent apparatus having a substantially tubular body comprising: an at least one braided segment and an at least one laser cut segment engaged with one another in series so as to form an elongate flow diverter stent; the at least one braided segment comprising: a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends; and the at least one laser cut segment comprising: a single piece of laser cut material having shape memory and super-elastic properties so as to form said laser cut segment capable of being inserted into a catheter and having a proximal end, a distal end, and a scaffold extending therebetween; the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; and the proximal end of said laser cut segment providing a plurality of engagement cells arranged circumferentially about the proximal end of said laser cut segment, each of the engagement cells configured for engagement with at least one strand of the at least one braided segment at the distal end of the at least one braided segment; whereby, during use of the apparatus, with the apparatus positioned within a catheter and moved to a target area of a patient's vascular system, each of the at least one braided segment and at least one laser cut segment is configured for moving into a shape memorized secondary shape capable of filling the target area as the apparatus exits the catheter.

2. The flow diverter stent apparatus according to embodiment 1, further comprising: a single braided segment; a first laser cut segment engaged with the distal end of the braided segment; and a second laser cut segment engaged with the proximal end of the braided segment.

3. The flow diverter stent apparatus according to embodiments 1-2, wherein the distal end of the second laser cut segment provides a plurality of engagement cells arranged circumferentially about the distal end of the second laser cut segment, each of the engagement cells configured for engagement with at least one strand of the at least one braided segment at the proximal end of the braided segment.

4. The flow diverter stent apparatus according to embodiments 1-3, wherein: each of the engagement cells of the second laser cut segment is an arrangement of four struts, wherein the struts are symmetrical along both a longitudinal axis and a circumferential axis of the second laser cut segment; and the struts of each of the engagement cells define a joint apex that is oriented substantially longitudinally relative to the second laser cut segment.

5. The flow diverter stent apparatus according to embodiments 1-4, wherein the joint apex of each engagement cell is configured for engagement with one or more of the strands of the proximal end of the braided segment.

6. The flow diverter stent apparatus according to embodiments 1-5, wherein the joint apex of each engagement cell is positioned so as to be in overlapping contact with and secured to a corresponding at least one strand of the braided segment at the proximal end of the braided segment.

7. The flow diverter stent apparatus according to embodiments 1-6, wherein each of the braided segment, first laser cut segment and second laser cut segment has a segment length that is equal to approximately one-third of an overall length of the apparatus.

8. The flow diverter stent apparatus according to embodiments 1-7, further comprising: a single laser cut segment; a first braided segment engaged with the proximal end of the laser cut segment; and a second braided segment engaged with the distal end of the laser cut segment.

9. The flow diverter stent apparatus according to embodiments 1-8, wherein the distal end of the laser cut segment provides a plurality of engagement cells arranged circumferentially about the distal end of the laser cut segment, each of the engagement cells configured for engagement with at least one strand of the second braided segment at the proximal end of the second braided segment.

10. The flow diverter stent apparatus according to embodiments 1-9, wherein: each of the engagement cells of the laser cut segment is an arrangement of four struts, wherein the struts are symmetrical along both a longitudinal axis and a circumferential axis of the laser cut segment; and the struts of each of the engagement cells define a joint apex that is oriented substantially longitudinally relative to the laser cut segment.

11. The flow diverter stent apparatus according to embodiments 1-10, wherein the joint apex of each engagement cell is configured for engagement with one or more of the strands of the proximal end of the second braided segment.

12. The flow diverter stent apparatus according to embodiments 1-11, wherein the joint apex of each engagement cell is positioned so as to be in overlapping contact with and secured to a corresponding at least one strand of the second braided segment at the proximal end of the second braided segment.

13. The flow diverter stent apparatus according to embodiments 1-12, wherein each of the laser cut segment, first braided segment and second braided segment has a segment length that is equal to approximately one-third of an overall length of the apparatus.

14. The flow diverter stent apparatus according to embodiments 1-13, further comprising: a single braided segment; and a single laser cut segment engaged with the distal end of the braided segment.

15. The flow diverter stent apparatus according to embodiments 1-14, wherein each of the braided segment and laser cut segment has a segment length that is equal to approximately one-half of an overall length of the apparatus.

16. The flow diverter stent apparatus according to embodiments 1-15, wherein the at least one braided segment has a segment length that is substantially the same as a segment length of the at least one laser cut segment.

17. The flow diverter stent apparatus according to embodiments 1-16, wherein the at least one braided segment has a segment length that is different than a segment length of the at least one laser cut segment.

18. The flow diverter stent apparatus according to embodiments 1-17, wherein each of the radial support cells have the same size, shape and dimensions.

19. The flow diverter stent apparatus according to embodiments 1-18, wherein one or more of the radial support cells have a different size, shape and/or dimensions.

20. The flow diverter stent apparatus according to embodiments 1-19, wherein each of the flexible support cells have the same size, shape and dimensions.

21. The flow diverter stent apparatus according to embodiments 1-20, wherein one or more of the flexible support cells have a different size, shape and/or dimensions.

22. The flow diverter stent apparatus according to embodiments 1-21, wherein: a center axis of each radial axial row and flexible axial row is parallel to a longitudinal axis of the at least one laser cut segment; each radial axial row is arranged so as to be offset along the longitudinal axis of the at least one laser cut segment from each adjacent radial axial row; and each radial support cell is formed from four of the plurality of interconnected struts of the scaffold which are arranged so as to be symmetrical along both the longitudinal axis and the circumferential axis of the at least one laser cut segment.

23. The flow diverter stent apparatus according to embodiments 1-22, wherein: each of the engagement cells of the at least one laser cut segment is an arrangement of four struts, wherein the struts are symmetrical along both a longitudinal axis and a circumferential axis of the at least one laser cut segment; and the struts of each of the engagement cells define a joint apex that is oriented substantially longitudinally relative to the at least one laser cut segment.

24. The flow diverter stent apparatus according to embodiments 1-23, wherein the joint apex of each engagement cell is configured for engagement with one or more of the strands of the corresponding distal end of the at least one braided segment.

25. The flow diverter stent apparatus according to embodiments 1-24, wherein the joint apex of each engagement cell is positioned so as to be in overlapping contact with and secured to a corresponding at least one strand of the at least one braided segment at the distal end of the at least one braided segment.

26. The flow diverter stent apparatus according to embodiments 1-25, wherein each of the engagement cells of the at least one laser cut segment have the same size, shape and dimensions.

27. The flow diverter stent apparatus according to embodiments 1-26, wherein one or more of the engagement cells of the at least one laser cut segment have a different size, shape and/or dimensions.

28. The flow diverter stent apparatus according to embodiments 1-27, wherein the engagement cells of the at least one laser cut segment are arranged circumferentially about the proximal end of the at least one laser cut segment so as to be equally spaced apart and symmetrical about the proximal end of the at least one laser cut segment.

29. The flow diverter stent apparatus according to embodiments 1-28, wherein the engagement cells of the at least one laser cut segment are arranged circumferentially about the proximal end of the at least one laser cut segment so as to be unequally spaced apart and asymmetrical about the proximal end of the at least one laser cut segment.

30. The flow diverter stent apparatus according to embodiments 1-29, wherein the engagement cells of the at least one laser cut segment are sized and configured for creating an at least one segment gap between the proximal end of the at least one laser cut segment and the distal end of the at least one braided segment.

31. The flow diverter stent apparatus according to embodiments 1-30, wherein the engagement cells of the at least one laser cut segment are sized and configured for positioning the proximal end of the at least one laser cut segment in substantially abutting contact with the distal end of the at least one braided segment.

32. The flow diverter stent apparatus according to embodiments 1-31, wherein one or more of the radial support cells of the at least one laser cut segment are sized for allowing a catheter to pass therethrough.

33. The flow diverter stent apparatus according to embodiments 1-32, wherein one or more of the radial support cells of the at least one laser cut segment are sized for allowing at least one further instance of the apparatus to pass therethrough.

34. The flow diverter stent apparatus according to embodiments 1-33, wherein one or more of the at least one segment gap is sized for allowing at least one further instance of the apparatus to pass therethrough.

35. The flow diverter stent apparatus according to embodiments 1-34, wherein each flexible support cell is formed from four of the plurality of interconnected struts of the scaffold which are arranged such that two adjacent flexible axial rows have mirror symmetry along the longitudinal axis of the at least one laser cut segment.

36. The flow diverter stent apparatus according to embodiments 1-35, wherein, for each of the radial support cells, the four struts that form said radial support cells are disposed such that a pair of opposing vertical struts are oriented substantially perpendicular to the longitudinal axis of the at least one laser cut segment.

37. The flow diverter stent apparatus according to embodiments 1-36, wherein the vertical struts of each radial support cell is configured for buckling inwardly toward one another when exposed to a radial force.

38. The flow diverter stent apparatus according to embodiments 1-37, wherein the vertical struts of each radial support cell are configured for deforming from a linear shape to a curved shape having a large radius when said vertical struts are exposed to the radial force.

39. The flow diverter stent apparatus according to embodiments 1-38, wherein the vertical struts of each radial support cell is configured for deform outwardly from one another when exposed to a tensile force while the at least one laser cut segment is in a longitudinally constrained configuration.

40. The flow diverter stent apparatus according to embodiments 1-39, wherein an angle between the struts is greater than 0° when the at least one laser cut segment is in the longitudinally constrained configuration.

41. The flow diverter stent apparatus according to embodiments 1-40, wherein a radial force exerted by the at least one laser cut segment is lower when the at least one laser cut segment is in the longitudinally constrained configuration than when the at least one laser cut segment is in each of an unconstrained configuration and a radially constrained configuration.

42. The flow diverter stent apparatus according to embodiments 1-41, wherein the radial force exerted by the at least one laser cut segment when the at least one laser cut segment is in the longitudinally constrained configuration permits the at least one laser cut segment to be compressed and loaded into a stent delivery system for insertion into a body lumen.

43. The flow diverter stent apparatus according to embodiments 1-42, further comprising an at least one radiopaque element formed on the at least one laser cut segment.

44. The flow diverter stent apparatus according to embodiments 1-43, wherein the at least one radiopaque element is positioned on one or both of the proximal end and distal end of the at least one laser cut segment.

45. The flow diverter stent apparatus according to embodiments 1-44, wherein the at least one radiopaque element is positioned on a segment length of the at least one laser cut segment.

46. The flow diverter stent apparatus according to embodiments 1-45, wherein the at least one radiopaque element is constructed out of at least one of a tungsten-loaded polymer, platinum, chromium, cobalt, tantalum, nitinol, gold, silver, bismuth subcarbonate, barium sulfate, bismuth oxychloride, bismuth trioxide, stainless steel or alloys thereof.

47. The flow diverter stent apparatus according to embodiments 1-46, wherein the apparatus is removably engageable with a delivery mechanism.

48. The flow diverter stent apparatus according to embodiments 1-47, wherein at least one of the strands of the at least one braided segment is comprised of an at least one material that is different from the other strands.

49. The flow diverter stent apparatus according to embodiments 1-48, wherein at least one of the strands of the at least one braided segment is comprised of an at least one radiopaque material.

50. The flow diverter stent apparatus according to embodiments 1-49, wherein at least one of the strands of the at least one braided segment is comprised of an at least one non-radiopaque material.

51. The flow diverter stent apparatus according to embodiments 1-50, wherein at least one of the strands of the at least one braided segment is comprised of both radiopaque and non-radiopaque materials.

52. The flow diverter stent apparatus according to embodiments 1-51, wherein at least one of the strands of the at least one braided segment is impregnated or coated with an at least one therapeutic.

53. The flow diverter stent apparatus according to embodiments 1-52, wherein the at least one braided segment defines a plurality of braid gaps between strands along a segment length of the at least one braided segment.

54. The flow diverter stent apparatus according to embodiments 1-53, wherein at least one of the strands of the at least one braided segment has a strand diameter that is different from the strand diameter of the other strands of the at least one braided segment.

55. The flow diverter stent apparatus according to embodiments 1-54, wherein the shape memorized secondary shape is at least one of a helical shape, a vortical shape, a flat spiral shape, a complex spiral shape, and a three-dimensional shape.

56. A flow diverter stent apparatus having a substantially tubular body comprising: a first laser cut segment, a braided segment, and a second laser cut segment engaged with one another in series so as to form an elongate flow diverter stent; the braided segment comprising: a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends; the first laser cut segment comprising: a single piece of laser cut material having shape memory and super-elastic properties so as to form said laser cut segment capable of being inserted into a catheter and having a proximal end, a distal end, and a scaffold extending therebetween; the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; and the proximal end of said laser cut segment providing a plurality of engagement cells arranged circumferentially about the proximal end of said laser cut segment, each of the engagement cells configured for engagement with at least one strand of the braided segment at the distal end of the braided segment; and the second laser cut segment comprising: a single piece of laser cut material having shape memory and super-elastic properties so as to form said laser cut segment capable of being inserted into a catheter and having a proximal end, a distal end, and a scaffold extending therebetween; the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; and the distal end of said laser cut segment providing a plurality of engagement cells arranged circumferentially about the distal end of said laser cut segment, each of the engagement cells configured for engagement with at least one strand of the braided segment at the proximal end of the braided segment; whereby, during use of the apparatus, with the apparatus positioned within a catheter and moved to a target area of a patient's vascular system, each of the first laser cut segment, braided segment, and second laser cut segment is configured for moving into a shape memorized secondary shape capable of filling the target area as the apparatus exits the catheter.

57. A flow diverter stent apparatus having a substantially tubular body comprising: a first braided segment, a laser cut segment, and a second braided segment engaged with one another in series so as to form an elongate flow diverter stent; each of the first and second braided segments comprising: a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends; and the laser cut segment comprising: a single piece of laser cut material having shape memory and super-elastic properties so as to form said laser cut segment capable of being inserted into a catheter and having a proximal end, a distal end, and a scaffold extending therebetween; the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; the proximal end of said laser cut segment providing a plurality of engagement cells arranged circumferentially about the proximal end of said laser cut segment, each of the engagement cells configured for engagement with at least one strand of the first braided segment at the distal end of the first braided segment; and the distal end of said laser cut segment providing a plurality of engagement cells arranged circumferentially about the distal end of said laser cut segment, each of the engagement cells configured for engagement with at least one strand of the second braided segment at the proximal end of the second braided segment; whereby, during use of the apparatus, with the apparatus positioned within a catheter and moved to a target area of a patient's vascular system, each of the first braided segment, laser cut segment, and second braided segment is configured for moving into a shape memorized secondary shape capable of filling the target area as the apparatus exits the catheter.

In closing, regarding the exemplary embodiments of the present invention as shown and described herein, it will be appreciated that a flow diverter stent apparatus is disclosed and configured for treating intracranial aneurysms. Because the principles of the invention may be practiced in a number of configurations beyond those shown and described, it is to be understood that the invention is not in any way limited by the exemplary embodiments, but is generally directed to a flow diverter stent apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art, or at least encompassing a range of plus or minus ten percent above and below the value of the stated characteristic, item, quantity, parameter, property, or term.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that any methods disclosed herein, along with the order in which the respective elements of any such method are performed, are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

All of the material in this patent document issue subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What is claimed is:

1. A flow diverter stent apparatus having a substantially tubular body comprising:
an at least one braided segment and an at least one laser cut segment engaged with one another in series so as to form an elongate flow diverter stent;
the at least one braided segment comprising:
a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends; and
the at least one laser cut segment comprising:
a single piece of laser cut material having shape memory and super-elastic properties so as to form said laser cut segment capable of being inserted into a catheter and having a proximal end, a distal end, and a scaffold extending therebetween;
the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; and
the proximal end of said laser cut segment providing a plurality of engagement cells arranged circumferentially about the proximal end of said laser cut segment, each of the engagement cells configured for engagement with at least one strand of the at least one braided segment at the distal end of the at least one braided segment;
whereby, during use of the apparatus, with the apparatus positioned within a catheter and moved to a target area of a patient's vascular system, each of the at least one braided segment and at least one laser cut segment is configured for moving into a shape memorized secondary shape capable of filling the target area as the apparatus exits the catheter.

2. The flow diverter stent apparatus of claim 1, further comprising:
a single braided segment;
a first laser cut segment engaged with the distal end of the braided segment; and
a second laser cut segment engaged with the proximal end of the braided segment.

3. The flow diverter stent apparatus of claim 1, further comprising:
a single laser cut segment;
a first braided segment engaged with the proximal end of the laser cut segment; and
a second braided segment engaged with the distal end of the laser cut segment.

4. The flow diverter stent apparatus of claim 1, further comprising:
a single braided segment; and
a single laser cut segment engaged with the distal end of the braided segment.

5. The flow diverter stent apparatus of claim 1, wherein each of the radial support cells have the same size, shape and dimensions.

6. The flow diverter stent apparatus of claim 1, wherein each of the flexible support cells have the same size, shape and dimensions.

7. The flow diverter stent apparatus of claim 1, wherein:
a center axis of each radial axial row and flexible axial row is parallel to a longitudinal axis of the at least one laser cut segment;
each radial axial row is arranged so as to be offset along the longitudinal axis of the at least one laser cut segment from each adjacent radial axial row; and
each radial support cell is formed from four of the plurality of interconnected struts of the scaffold which are arranged so as to be symmetrical along both the longitudinal axis and the circumferential axis of the at least one laser cut segment.

8. The flow diverter stent apparatus of claim 1, wherein:
each of the engagement cells of the at least one laser cut segment is an arrangement of four struts, wherein the struts are symmetrical along both a longitudinal axis and a circumferential axis of the at least one laser cut segment; and
the struts of each of the engagement cells define a joint apex that is oriented substantially longitudinally relative to the at least one laser cut segment.

9. The flow diverter stent apparatus of claim 8, wherein the joint apex of each engagement cell is configured for engagement with one or more of the strands of the corresponding distal end of the at least one braided segment.

10. The flow diverter stent apparatus of claim 9, wherein the joint apex of each engagement cell is positioned so as to be in overlapping contact with and secured to a corresponding at least one strand of the at least one braided segment at the distal end of the at least one braided segment.

11. The flow diverter stent apparatus of claim 1, wherein the engagement cells of the at least one laser cut segment are sized and configured for creating an at least one segment gap between the proximal end of the at least one laser cut segment and the distal end of the at least one braided segment.

12. The flow diverter stent apparatus of claim 11, wherein one or more of the at least one segment gap is sized for allowing at least one further instance of the apparatus to pass therethrough.

13. The flow diverter stent apparatus of claim 1, wherein one or more of the radial support cells of the at least one laser cut segment are sized for allowing at least one further instance of the apparatus to pass therethrough.

14. The flow diverter stent apparatus of claim 1, wherein one or more of the radial support cells of the at least one laser cut segment are sized for allowing a catheter to pass therethrough.

15. The flow diverter stent apparatus of claim 1, further comprising an at least one radiopaque element formed on the at least one laser cut segment.

16. The flow diverter stent apparatus of claim 1, wherein at least one of the strands of the at least one braided segment is comprised of an at least one radiopaque material.

17. The flow diverter stent apparatus of claim 1, wherein the at least one braided segment defines a plurality of braid gaps between strands along a segment length of the at least one braided segment.

18. The flow diverter stent apparatus of claim 1, wherein the shape memorized secondary shape is at least one of a helical shape, a vortical shape, a flat spiral shape, a complex spiral shape, and a three-dimensional shape.

19. A flow diverter stent apparatus having a substantially tubular body comprising:
a first laser cut segment, a braided segment, and a second laser cut segment engaged with one another in series so as to form an elongate flow diverter stent;
the braided segment comprising:
a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends;
the first laser cut segment comprising:
a single piece of laser cut material having shape memory and super-elastic properties so as to form said laser cut segment capable of being inserted into a catheter and having a proximal end, a distal end, and a scaffold extending therebetween;
the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; and
the proximal end of said laser cut segment providing a plurality of engagement cells arranged circumferentially about the proximal end of said laser cut segment, each of the engagement cells configured for engagement with at least one strand of the braided segment at the distal end of the braided segment; and
the second laser cut segment comprising:
a single piece of laser cut material having shape memory and super-elastic properties so as to form said laser cut segment capable of being inserted into a catheter and having a proximal end, a distal end, and a scaffold extending therebetween;
the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment; and
the distal end of said laser cut segment providing a plurality of engagement cells arranged circumferentially about the distal end of said laser cut segment, each of the engagement cells configured for engagement with at least one strand of the braided segment at the proximal end of the braided segment;
whereby, during use of the apparatus, with the apparatus positioned within a catheter and moved to a target area of a patient's vascular system, each of the first laser cut segment, braided segment, and second laser cut segment is configured for moving into a shape memorized secondary shape capable of filling the target area as the apparatus exits the catheter.

20. A flow diverter stent apparatus having a substantially tubular body comprising:
a first braided segment, a laser cut segment, and a second braided segment engaged with one another in series so as to form an elongate flow diverter stent;
each of the first and second braided segments comprising:
a plurality of individual strands braided together so as to form said braided segment having a proximal end and an opposing distal end, said braided segment forming an elongated primary shape capable of being inserted into a catheter and having an inner diameter along an entire segment length thereof between the proximal and distal ends; and
the laser cut segment comprising:
a single piece of laser cut material having shape memory and super-elastic properties so as to form said laser cut segment capable of being inserted into a catheter and having a proximal end, a distal end, and a scaffold extending therebetween;
the scaffold having a plurality of interconnected struts defining a plurality of radial axial rows of radial support cells and a plurality of flexible axial rows of flexible support cells, such that the plurality of radial axial rows and the plurality of flexible axial rows are disposed in an alternating pattern along a circumferential axis of said laser cut segment;
the proximal end of said laser cut segment providing a plurality of engagement cells arranged circumferentially about the proximal end of said laser cut segment, each of the engagement cells configured for engagement with at least one strand of the first braided segment at the distal end of the first braided segment; and
the distal end of said laser cut segment providing a plurality of engagement cells arranged circumferentially about the distal end of said laser cut segment, each of the engagement cells configured for engagement with at least one strand of the second braided segment at the proximal end of the second braided segment;
whereby, during use of the apparatus, with the apparatus positioned within a catheter and moved to a target area of a patient's vascular system, each of the first braided segment, laser cut segment, and second braided segment is configured for moving into a shape memorized secondary shape capable of filling the target area as the apparatus exits the catheter.
